(19) United States
(12) Patent Application Publication
(10) Pub. No.: US 2024/0120108 A1
(43) Pub. Date: Apr. 11, 2024

(54) MACHINE LEARNING METHOD FOR ENHANCING CARE OF A PATIENT USING VIDEO AND AUDIO ANALYTICS

(71) Applicant: Insight Direct USA, Inc., Chandler, AZ (US)

(72) Inventors: Michael Griffin, Wayland, MA (US); Hailey Kotvis, Wauwatosa, WI (US); Josephine Miner, Hope, RI (US); Porter Moody, Wayland, MA (US); Kayla Poulsen, Natick, MA (US); Austin Malmin, Gilbert, AZ (US); Sarah Onstad-Hawes, Seattle, WA (US); Gloria Solovey, Arlington, MA (US); Austin Streitmatter, Palm Harbor, FL (US)

(21) Appl. No.: 18/463,685

(22) Filed: Sep. 8, 2023

Related U.S. Application Data
(60) Provisional application No. 63/414,198, filed on Oct. 7, 2022.

(51) Int. Cl. G16H 50/30; G06T 7/00; G06V 10/774; G06V 20/40; G10L 15/02; G10L 15/06; G10L 15/18; G10L 25/66

(52) U.S. Cl. CPC G16H 50/30; G06T 7/0012; G06V 10/774; G06V 20/41; G06V 20/46; G10L 15/02; G10L 15/063; G10L 15/1815; G10L 25/66; G16H 10/60

(57) ABSTRACT

Apparatus and associated methods relate to enhancing care of a patient using video and audio analytics. Video data, audio data, and semantic text data are extracted from a video stream of the patient. The video data are analyzed to identify a first feature set. The audio data are analyzed to identify a second feature set. The semantic text data are analyzed to identify a third feature set. Using a computer-implemented machine-learning model, a health outcome of the patient is predicted based on the first, second, and/or third features sets. The health outcome that is predicted is compared with the set of health outcomes of the training patients classified with the patient classification of the patient. Differences are identified between the feature sets corresponding to the patient and feature sets of the training patients who have better health outcomes the patient's predicted health outcome. The differences identified are then reported.

### CROSS-REFERENCE TO RELATED APPLICATION

The present application claims priority to U.S. provisional patent application Ser. No. 63/414,198 by M. Griffin, filed Oct. 7, 2022 and entitled “A MACHINE LEARNING METHOD FOR ENHANCING CARE OF A PATIENT USING VIDEO AND AUDIO ANALYTICS.

### BACKGROUND

Video cameras are used in a great many places to accomplish many different things. Video cameras are deployed in our cities, along our highways and roads, inside and outside homes and buildings, inside and outside of vehicles, etc. Such cameras can be used to provide surveillance, to enforce traffic laws, to help identify guests and visitors, to assist automatic navigation of vehicles, etc. Image processors can operate on such video streams to enhance the video stream and/or to measure metrics pertaining to the video stream. These metrics can be used to provide various capabilities not provided by the video stream alone.

Heretofore, the capabilities provided by image processing algorithms were fixed by the programmer's code. Improvements can be made to such capabilities using machine learning. Machine learning can also be used to provide entirely new capabilities that have not previously been provided.

### SUMMARY

Some embodiments relate to a method for enhancing care for a patient. In the method, video data, audio data, and semantic text data is extracted from a video stream configured to capture the patient. The video data is analyzed to identify a first feature set of video features identified by a computer-implemented machine-learning engine as being indicative of at least one of a set of health outcomes of training patients classified with the patient classification of the patient. The audio data is analyzed to identify a second feature set of audio features identified by the computer-implemented machine-learning engine as being indicative of at least one of the set of health outcomes of the training patients classified with the patient classification of the patient. The semantic text data is analyzed to identify a third feature set of semantic text features identified by the computer-implemented machine-learning engine as being indicative of set of at least one of the set of health outcomes of the training patients classified with the patient classification of the patient. A predicted health outcome of the patient is predicted based on the first, second and/or third features sets. The patient's behavior is determined using a computer-implemented machine-learning model generated by the computer-implemented machine-learning engine. The predicted health outcome of the patient is predicted using a computer-implemented machine-learning model generated by the computer-implemented machine-learning engine. The predicted health outcome is compared with a set of known training health outcomes of the training patients classified with the patient classification of the patient. Differences between the first, second, and/or third feature sets corresponding to the patient are compared with and feature sets of the training patients who have better health outcomes than the predicted health outcome of the patient. The differences identified are reported.

Some embodiments relate to a system for enhancing care for a patient. The system includes a video camera configured to capture a video stream of a patient, a processor configured to receive the video stream of the patient, and computer readable memory. The computer readable memory is encoded with instructions that, when executed by the processor, cause the system to perform the following steps. The system extracts video data, audio data, and semantic text data from a video stream configured to capture patient. The system analyzes the video data to identify a first feature set of video features identified by a computer-implemented machine-learning engine as being indicative of at least one of a set of health outcomes of training patients classified with the patient classification of the patient. The system analyzes the audio data to identify a second feature set of audio features identified by the computer-implemented machine-learning engine as being indicative of at least one of the set of health outcomes of the training patients classified with the patient classification of the patient. The system analyzes the semantic text data to identify a third feature set of semantic text features identified by the computer-implemented machine-learning engine as being indicative of set of at least one of the set of health outcomes of the training patients classified with the patient classification of the patient. The system predicts the predicted health outcome of the patient based on the first, second and/or third features sets, wherein the predicted health outcome of the patient is predicted using a computer-implemented machine-learning model generated by the computer-implemented machine-learning engine. The system compares the predicted health outcome with a set of known training health outcomes of the training patients classified with the patient classification of the patient. The system identifies differences between the first, second, and/or third feature sets corresponding to the patient and feature sets of the training patients who have better health outcomes than the predicted health outcome of the patient. The system reports the differences identified.

### BRIEF DESCRIPTION OF THE DRAWINGS

FIG. 1 is a block diagram of a patient monitoring system that determines metrics of a patient based on a video stream.

FIG. 2A is a perspective view of a patient room in which a patient-monitoring system is monitoring patient behavior.

FIG. 2B is a block diagram of a patient-monitoring system that is monitoring patient behavior.

FIG. 3 is a flowchart for a method for assessing a confidence level of verbal statements expressed by a person during a verbal communication.

FIG. 4 is a flowchart of a method for automatically invoking an alert when a patient's behavior is an alerting behavior.

FIG. 5 is a flowchart of a method for automatically invoking an alert when a patient's behavior, as determined by audio data and semantic text data, is an alerting behavior.

FIG. 6 is a flowchart of a method for predicting a health outcome for a video-monitored patient.

FIG. 7 is a flowchart of a method for recommending changes in the care for a video-monitored patient.

### DETAILED DESCRIPTION

To advance the useful arts with regard to use of video streams, computer-implemented machine learning can be exploited. Computer-implemented machine learning can greatly assist the development of methods and models that can identify, determine, predict, and/or assess various metrics that heretofore have proved difficult to identify, determine, predict, and/or assess from video data alone or from video data in combination with other data. Such machine learning can greatly improve the capabilities of video analytics—the use of data extracted from a video stream. This specification will disclose various new capabilities and the methods of achieving such capabilities using such a combination of video streams along with computer-implemented machine learning. Such new capabilities include such things as: i) determining a behavior of a video-monitored person; ii) automatically invoking an alert when a patient's behavior is an alerting behavior; iii) assessing a confidence level of verbal communications of a video-monitored person; iv) predicting a health outcome for a video-monitored patient; v) recommending changes in the care for a video-monitored patient, etc. Although some of these methods can be performed on persons in various settings, many of the examples described below will be set in a medical care facility with the methods being performed on a patient.

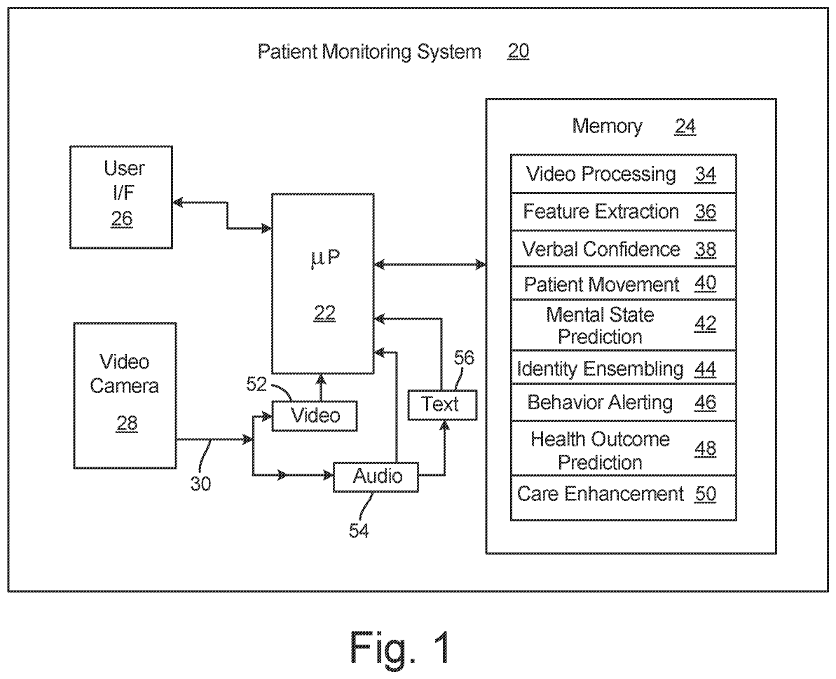

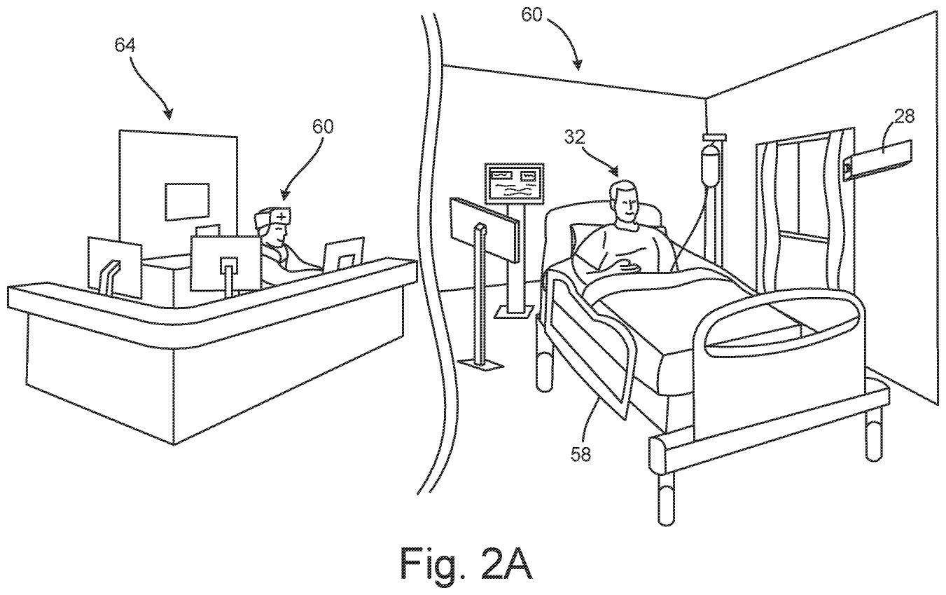

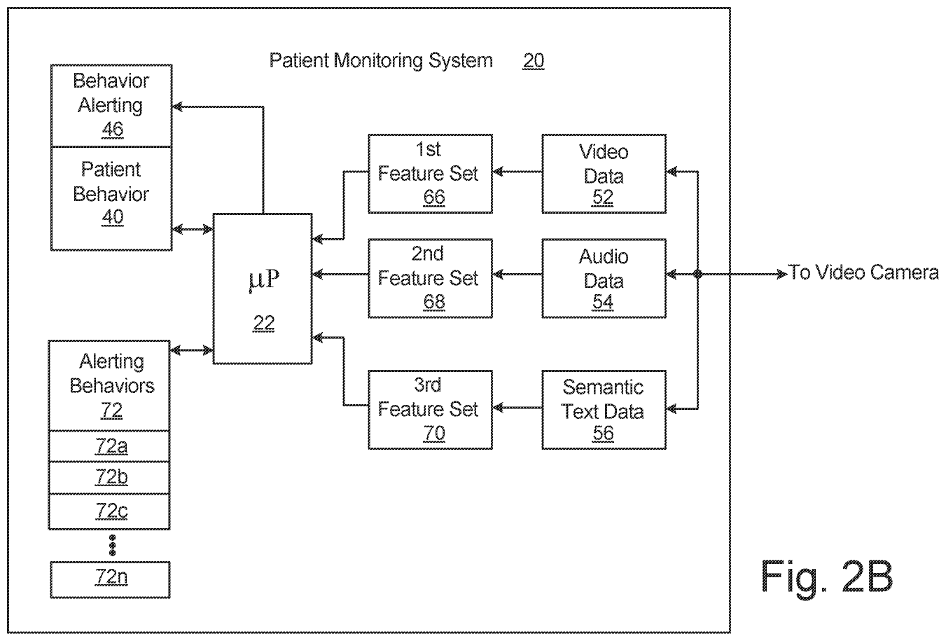

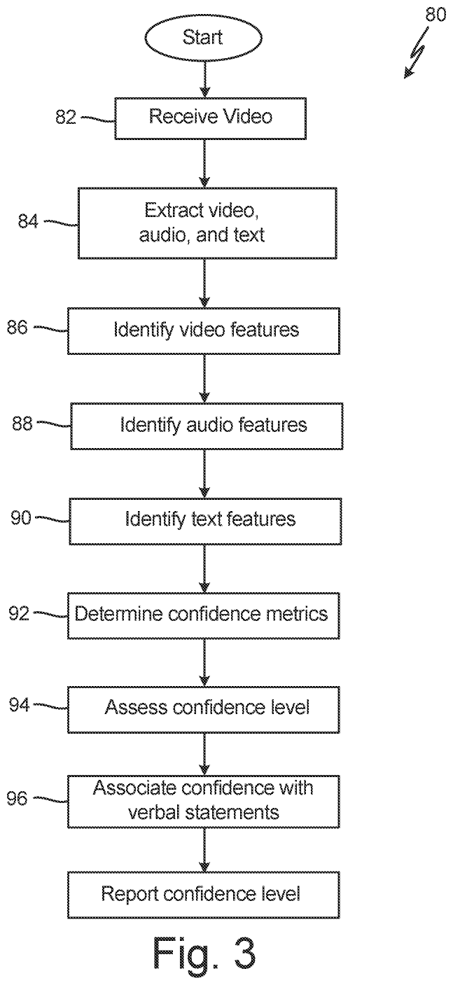

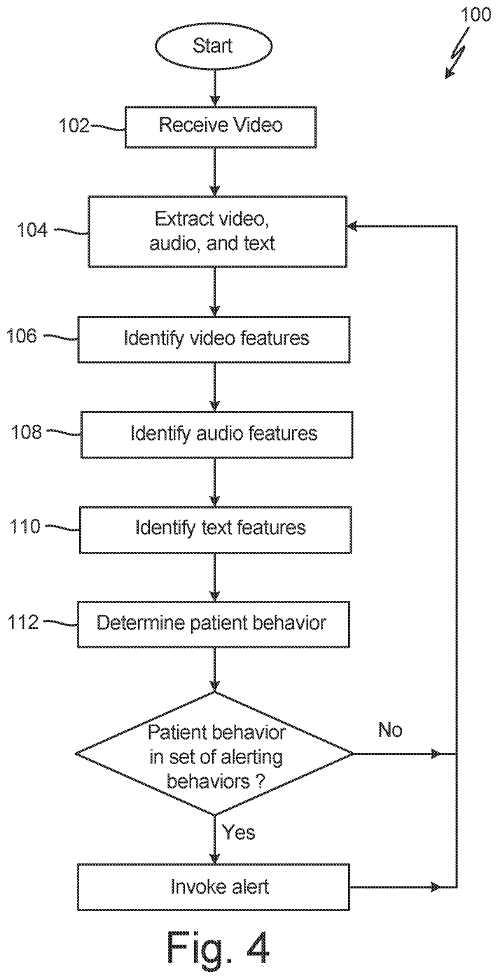

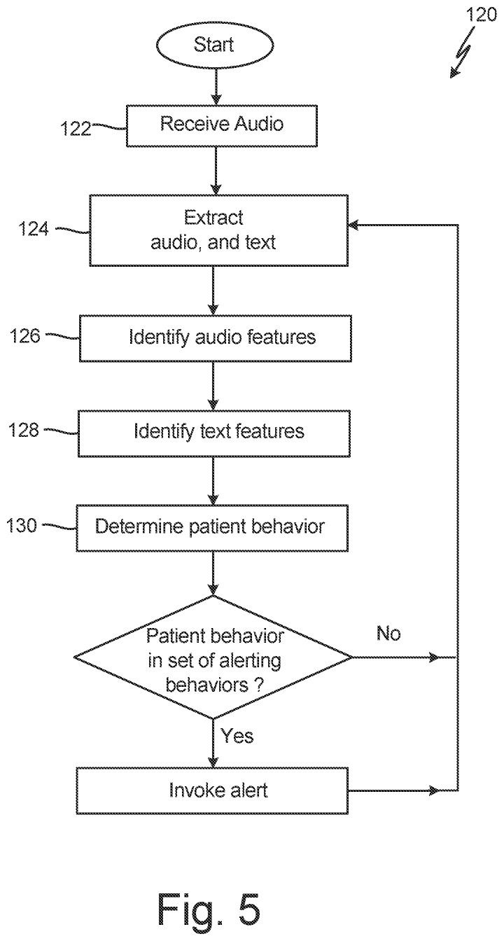

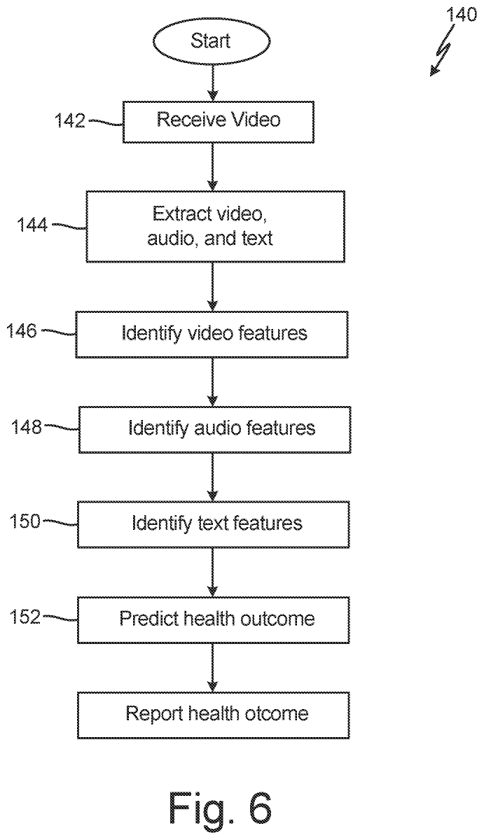

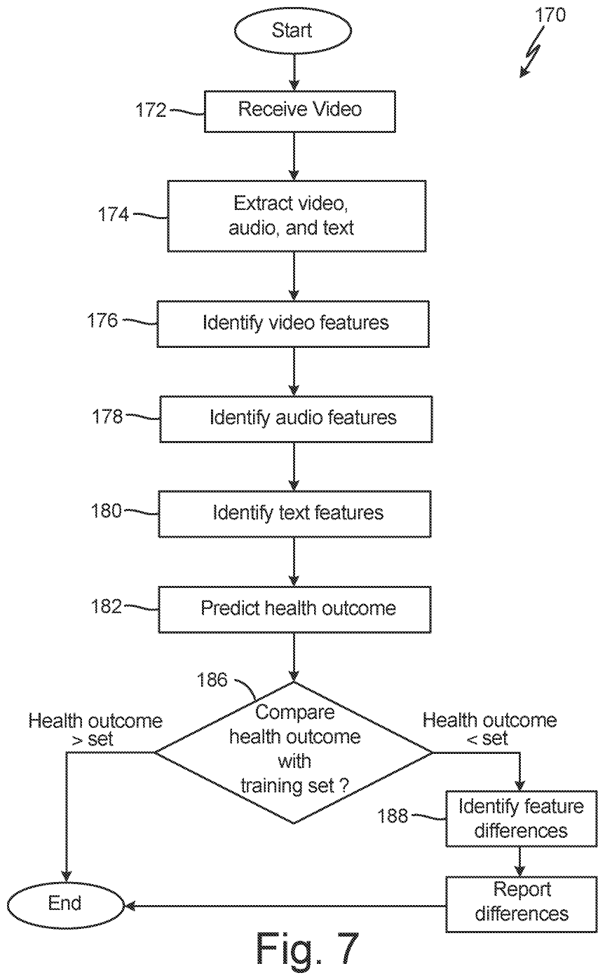

This specification is organized as follows. First, a general survey of some of the capabilities that can be obtained using computer-implemented machine-learning models operating on video streams will be presented. Next, an example system that uses such computer-implemented machine-learning models to perform these various capabilities will be described with reference to FIG. 1. Then, a specific example of one such method—automatically generating an alert based on a patient's behavior determined using a computer-implemented machine-learning model—will be described with reference to FIGS. 2A and 2B. Then follows discussions of various functional capabilities of using machine learning in conjunction with a video stream or an audio stream. A method for assessing a confidence level of verbal communications of a patient will be described with reference to FIG. 3. A method for automatically invoking an alert when a patient's behavior is an alerting behavior, as determined from a video stream, will be described with reference to FIG. 4. A method for automatically invoking an alert when a patient's behavior is an alerting behavior, as determined from an audio stream, will be described with reference to FIG. 5. A method for predicting a health outcome for a video-monitored patient will be described with reference to FIG. 6. Finally, a method for enhancing care for a patient will be described with reference to FIG. 7.

General Overview of Computer-Implemented Machine Learning Methods

Healthcare facilities often monitor a patient's vital signs so as to provide the patient's care providers up-to-date status of the patient's health condition. Various vital signs or health metrics are routinely monitored, such as, for example, heart rate, body temperature, respiratory rate, and arterial blood pressures. For birthing mothers, hospitals monitor various other health metrics of health conditions of both the mother and the child. Such health metrics are provided to doctors and nurses, who can then respond to such monitored health metrics that indicate such a response appropriate.

Hospitals are usually also equipped with security cameras, especially in areas where security breaches are most often committed, such as, for example at entrances, public waiting areas, and in emergency rooms. Such security cameras are primarily used for security monitoring purposes. The prices of these cameras have plummeted, at least in part, as a result of the market growth of such security cameras. Such pricing and availability of these cameras provide opportunities for using cameras for various other purposes in a healthcare facility.

Although a patient's vital signs are routinely monitored by healthcare providers, many other patient metrics have not been routinely monitored. Some of these previously unmonitored metrics could, if measured, be indicative of the patient's health condition, and therefore used to enhance care for the patient. Metrics of patient behaviors are some such metrics that can be indicative of a patient's health condition. Various patient behaviors can be captured in a video stream from a video camera mounted in the patient's room. Many of the patient's behaviors captured can be indicative of the patient's health and/or assessed as being helpful or harmful to the patient's health outcome (e.g., to the patient's recovery). Such patient behaviors can include various distinct categories of behaviors. For example, physical movement or lack thereof, mental state, verbal communications, and non-verbal sounds are categories of patient behaviors that could be used to enhance care of the patient. Such patient behaviors have not been significantly leveraged to enhance care for the patient, at least in part, due to the difficulty of obtaining objective metrics of such patient behaviors. For example, such patient behaviors, although captured in a video stream, have proved difficult to automatically classify. Using machine learning, as will be described below, such metrics and/or classifications can now automatically be determined and so quickly obtained (e.g., in real time).

A Patient's Movements

A patient's movements or actions can be indicative of a patient's physical condition. For example, if a patient exhibits difficulty in sitting or walking, such difficulty can be indicative of a stage of recovery or lack thereof. A computer-implemented machine-learning model can be generated and used to automatically characterize such patient movements. Then, such characterizations of the patient's movements can be determined to be consistent or inconsistent with a doctor's orders. For example, if the doctor orders a patient to ask for assistance if he/she would want to walk, a patient's attempt to walk by him/herself can be immediately classified and an alert can be invoked, thereby permitting a care provider to intercept the patient's attempt and to provide assistance thereto, for example.

A Patient's Non-Verbal Communications

A patient's non-verbal communications can be helpful to doctors, nurses, and other healthcare providers in various ways. For example, such non-verbal communications can be indicative of the patient's pain level, discomfort, etc. If, for example, a patient is moaning, wailing, groaning, etc., such non-verbal communication can indicate a high level of pain sensation. Non-verbal communication can also be indicative of a patient's emotional state. If, for example, if a patient is crying, sniffling, weeping, etc., such non-verbal communication can indicate a patient's sadness, depression, grief, etc. After capture in a video stream, a computer-implemented machine-learning model can be used to automatically characterize the patient's non-verbal communications. Then, such characterizations of the patient's physical and/or emotional conditions can be reported to the care provider, who then can use such data to improve treatment of the patient, for example.

A Patient's Verbal Communications

A patient's verbal communications can be helpful to doctors, nurses, and other healthcare providers in various ways. For example, a patient's perception of his/her own condition (e.g., sense of pain, sense of nausea, sense of dizziness, sense of strength, etc.) can be helpful for diagnostic purposes as well as for evaluating success or failure of treatments provided for the patient. After capture in a video stream, a computer-implemented machine-learning model can be used to automatically characterize the patient's verbal communications, as being indicative of his/her perceived condition or not. Then, such patient perception of his/her own condition can be reported to the health care staff, who then can use such data to improve treatment of the patient.

Confidence Level of a Patient's Verbal Communications

Verbal communications, however, are only as good as such verbal communications are in accordance with reality. For example, a patient's verbal communications regarding a health condition are only as good as such verbal communications are in accordance the actual health condition of the patient. For various reasons (e.g., out of embarrassment, pride, fear, etc.), some patients' verbal communications might not be precisely aligned with the patients' actual health conditions. Using machine learning, confidence levels of a patient's verbal communications can be automatically assessed. Such confidence levels can be used alongside the patient's verbal communications so as to provide healthcare professionals with better understanding of these communications and therefore of the patient's health condition.

Predicting a Mental State of a Patient

Machine learning can be used at various levels so as ultimately to enhance care of patients. For example, machine learning can be used to generate a mental-state model that predicts a patient's mental state (e.g., tiredness, sleepiness, serenity, satisfaction, calmness, relaxation, contentment, distress, frustration, anger, annoyance, tension, fear, alarm, misery, sadness, depression, gloom, boredom, anguish, astonishment, amusement, excitement, happiness, delight, gladness, pleasure, thankfulness, gratitude, confusion, smugness, deliberation, anticipation, cheer, sympathy, trust, humor, envy, melancholy, hostility, resentment, revulsion, and/or ennui). Such mental states include mental states that are indicative of the patient's physical condition (e.g., tiredness, sleepiness, distress, etc.), mental states that are indicative of the patient's emotional state (e.g., delight, depression, anger, etc.), as well as mental states that can be indicative of either the patient's physical condition or the patient's emotional state (e.g., tension, misery, pleasure, etc.). Such a mental-state model can include a general mental-state model that is trained using a substantial number of different video streams or audio streams of a corresponding substantial number of different people exhibiting various mental states. Some mental-state models can include a patient-specific mental-state model that is trained using a video stream or an audio stream of the specific patient. Such patient-specific mental-state models can augment a general mental-state model, improving performance as the patient-specific mental-state model learns the specific mental states of the corresponding specific patient.

Automatically Invoking an Alert Based on Patient Behaviors

A patient's behaviors can be used in a variety of ways. For example, the patient's behaviors can be reported in a patient record, and/or can be compared with a set of alerting behaviors corresponding to a patient classification of the patient. Some such alerting behaviors might include: a patient falling; a patient exhibiting confusion; a patient behaving in a manner indicative of the patient experiencing pain; a patient's excessive coughing; a patient choking; a patient reaching for an assistance request button; a patient verbally asking for help; a patient crying; a patient moving erratically; a patient attempting to stand or walk; a patient behaving in a manner consistent with a mental state of depression; etc. Such a set of alerting behaviors can be different for different patient classifications. For instance, some behaviors can be considered to be alerting behaviors for patients of one classification, while not be considered alerting behaviors for patients of another classification. When the patient's behavior is found to be included in the set of alerting behaviors corresponding to the classification of the patient being monitored, an alert can be automatically invoked so as to notify a healthcare worker of the alerting behavior of the monitored patient.

Such alerts can be automatically invoked for other reasons as well. For example, identities of persons captured in the video stream can be ensembled (or otherwise aggregated) according to features extracted from the video stream or audio stream. The ensembled identities can be compared with a list of unauthorized visitors. When an identity, as ensembled according to the features extracted from the video stream or audio stream, is determined to be included in the list of unauthorized visitors, an alert can be automatically invoked so as to notify a healthcare worker and/or a security officer of the unauthorized visitor. In another example, metrics of the environmental condition of the patient's room can be compared with a set of alerting conditions. When the environmental condition is determined to be included in the set of alerting conditions, an alert can be automatically invoked so as to notify a staff member of the alerting condition.

Predicting a Patient's Health Outcome

Doctors have used traditional health data (e.g., diagnosed condition, heart rate, body temperature, respiratory rate, arterial blood pressures, lab results, medications administered, etc.), to predict the likely health outcomes of patients. Such predictions can be improved using other metrics obtained from video streams of these patients. For example, metrics of patient behaviors, as well as confidence levels of verbal communications, can be indicators of the patient's condition and therefore can be used to predict a patient's health outcome.

Other useful metrics can be obtained from the video stream as well. For example, patients' interactions with visitors and/or healthcare providers can also be indicative of the patients' conditions and/or be assessed as being helpful or harmful to the patient's health outcome. Therefore, such patient interactions can be used to improve prediction of the patients' health outcomes as well. Such patients' interactions can include, for example, patients' interactions with visitors, doctors, nurses, and other staff members. Furthermore, environmental conditions also can affect the health of patients. Therefore, such environmental conditions also can be used to predict the patients' health outcomes. Such environmental conditions can include, for example, lighting conditions, room temperature, room humidity, and/or a sound condition within the patients' rooms. Some of these environmental conditions, such as lighting and sound conditions, are captured in the video stream, and metrics can be automatically generated therefrom.

The various above-described metrics can be used to build a predictive model of patients' health outcomes. Machine learning can be used to identify metrics that are useful for predicting health outcomes and to create a health-outcome predictive model, which can be used on future patients to be monitored, as will be further described below. Such useful metrics can be identified, and such a health-outcome predictive model can be created by a computer-implemented machine-learning engine. The predicted health outcome of a patient can then be reported to the patient's doctor or other clinician, so that the prediction can be used in forming a care plan, for example. The predicted health outcome of a patient can be reported to the hospital, as well. The hospital can then use such predictions, for example, to better determine/predict such things as: time of patient discharge from the hospital; hospital staffing requirements; likelihood of the patient's return to the hospital, etc.

Enhancing Care of a Patient

Such a health-outcome predictive model also can be used to enhance care of the patient. After such metrics as described above are collected and the health-outcome predictive models are trained and evaluated, machine learning can be used to identify specific care modifications that are likely to contribute to recovery for patients of various patient classifications. The health outcomes predicted by the health-outcome predictive model can be used to enhance care for the patient so that the health outcome, as predicted by the health-outcome predictive model, can be realized. For example, if a patient has recovered from his/her illness but is identified as having depression with a high probability of requiring additional hospitalization within several weeks, the patient could be referred to a psychiatrist or prescribed anti-depression medication.

Some patient behaviors, patient interactions, and/or environmental conditions can contribute to improvement of patients' health outcomes for certain classifications of patients, and yet be found not to contribute to improvement of patients' health outcomes for other classifications of patients. Thus, using such metrics in conjunction with the classification of the patient can improve the utility of these metrics. A patient's classification can include diagnosed condition as well as patient attributes (e.g., bodyweight, other health conditions, medications, etc.). Machine learning can be used to determine how such patient behaviors, patient interactions, and/or environmental conditions affect patients' health outcomes for a variety of classifications of patients. Using such machine-learned relationships between these metrics obtained from the video stream or audio stream and predicted health outcomes, care for the patient can be enhanced by providing healthcare professionals with suggested responses to various of these metrics.

Training of Computer-Implemented Machine-learning engine

Training of computer-implemented machine-learning engines can be performed using a substantial number of training video streams of a corresponding substantial number of training patients. The numbers of training patients needed to train a computer-implemented machine-learning engine depends on the particular function that is to be performed by the computer implemented machine-learning engine. Regardless of the particular function, the number of video streams (and patients) should span the desired functional outputs so that every desired functional output can be generated under conditions calling for such a desired output. Typically, as more training patients are used to train a computer-implemented machine-learning engine, more outputs are possible, and determination of such outputs become more accurate. In essence, the computer-implemented machine-learning engine generates a computer-implemented machine-learning model that can determine (e.g., predict, assess, etc.) the desired function (e.g., determine patient behavior, assess confidence level of verbal communications, etc). The computer-implemented machine-learning model is based upon extracted features that the computer-implemented machine-learning engine has found to be indicative of the desired functional output. These features can be static features, as well as dynamic features (e.g., time between features, frequency of features, change from one feature to another, such as those that indicate movements, etc.). The computer-implemented machine-learning engine can combine features to create additional features. Such created features can include feature combinations of at least two of: a video feature, an audio feature, and a semantic text feature. Such a created features can include metrics related to: a number of times feature combination occurs; a frequency of occurrences of the feature combination; a time period between occurrences of the feature combination; and/or a time period between occurrences of a first feature combination and a second feature combination, for example.

The computer-implemented machine-learning engine generates coefficients of a computer-implemented machine-learning model, which are used to generate the functional outputs. In some embodiments, the computer-implemented machine-learning engine generates the form of the computer-implemented machine-learning model itself, as well as determining the coefficients thereof. The computer-implemented machine-learning engine receives the training video streams along with corresponding known functional outputs. For example, if the functional output of a machine-learning engine is to characterize patient movements, the training video streams will depict patients moving in various manners, and the known outputs will be the actual movements of the patients. Thus, the movements depicted in the video stream will effectively be annotated by the known actual movements of the patients.

The computer-implemented machine-learning engine will then extract features from the video data of the video stream and build a computer-implemented machine-learning model based on such extracted features. For example, the computer-implemented machine-learning engine can have static patient positions SP₁-SP_{M} as functional outputs. In addition to these static patient positions, the computer-implemented machine-learning engine can have dynamic patient movements DM₁-DM_{N} as functional outputs. The training of the computer-implemented machine-learning engine can be performed in a single phase or using multiple phases. For example, the computer-implemented machine-learning engine can first train on the video streams to generate the model for determining a patient's static position, and then train on the time sequence of such determined static positions to generate a model that determines a patient's movements.

Regardless of whether a single training phase or multiple training phases are used, each training phase is, in effect, an exercise in determining model coefficients of a computer-implemented machine-learning model. The model coefficients are determined so as to improve a correlation between the known functional outputs and the functional outputs as determined by the computer-implemented machine-learning model. Determination of model coefficients that improve, if not optimize, such a correlation can be performed by various regression techniques. If, for example, the model permits linear regression of the model coefficients, a linear regression can be performed so as to obtain optimal correlation between the known functional outputs and those functional outputs determined by the model using the video streams without using the known functional outputs.

Usually, some terms of the computer-implemented machine-learning model will be more heavily weighted (i.e., such terms have a model coefficient with a greater magnitude) than other terms. New terms can be made using various mathematical combinations of terms. For example, a ratio of two terms can be added to the model and a model coefficient for this new term can be obtained by training the new model using the training video streams. Any mathematical operation can be used to obtain new terms that combine other terms (e.g., multiplication, division, logarithms, exponentiation, etc.). Thus, the computer-implemented machine-learning engine generates the computer-implemented machine-learning model. And it does so based on a substantial number of training video streams that have known functional outputs.

Such a computer-implemented machine-learning model, as generated as described above, can be called a general computer-implemented machine-learning model because it was generated using many training video streams, none of which being a video stream of the specific patient for which the functional will be generated. Thus, the general computer-implemented machine-learning model has general model coefficients. Such a general computer-implemented machine-learning model can be augmented by a specific computer-implemented machine-learning model, which learns the functional outputs of the specific patient for whom the functional outputs are being determined. For example, a health care provider can watch the video stream of the specific patient and annotate the movements depicted therein. In doing so, the computer-implemented machine-learning model can then generate patient-specific model coefficients for the computer-implemented machine-learning model. In other embodiments, the video streams of the patient can augment the training video streams, and the computer-implemented machine-learning model can be retrained using this augmented set of training video streams.

In some embodiments, the terms that are very lightly weighted (i.e., those that have very small magnitude coefficients) can be discarded, so as to simplify the computer-implemented machine-learning model. This can permit the computer-implemented machine learning engine to extract fewer features from the video data, for example, when performing its function on a patient. Features identified as being useful by the computer-implemented machine-learning engine (i.e., those used in terms that have large magnitude coefficients) can then be extracted from the video data. The computer-implemented machine-learning engine is thus used in two ways: i) in a training manner to generate the computer-implemented machine-learning model; and ii) in real-time operation, extracting features from a video stream and applying such extracted features to the computer-implemented machine-learning model generated so as to obtain functional outputs (e.g., determining patient movements).

### Example System for Performing the Various Capabilities

FIG. 1 is a block diagram of a patient monitoring system that determines metrics of a patient using features extracted from a video stream. In FIG. 1, patient-monitoring system 20 includes processor 22, computer-readable memory 24, and user interface 26 and is connected to video camera 28. Video camera 28 is configured to generate video stream 30 of images taken of objects in the field of view of video camera 28. Computer-readable memory 24 is encoded with instructions that, when executed by processor 22, cause patient-monitoring system 20 to generate various metrics of patient 32 (depicted in FIG. 2A), and of the patient's surroundings. Computer-readable memory 24 can include software modules that enable patient-monitoring system 20 to determine such metrics, such as, for example: physical movement or lack thereof, mental state, verbal communications, confidence levels of such verbal communications, non-verbal sounds, persons captured in video stream 30, patient interactions, environmental condition, etc. To determine these and other metrics, computer-readable memory 24 includes video-processing module 34, feature-extraction module 36, verbal-confidence module 38, patient-movement module 40, mental-state prediction module 42, identity-ensembling module 44, behavior-alerting module 46, health-outcome prediction module 48, and care-enhancement module 50.

First, some possible hardware configurations of patient-monitoring system 20 will be described, and then the various software modules will be described. Processor 22 is configured to execute software, applications, and/or programs stored in computer-readable memory 24. Examples of processor 22 can include one or more of a processor, a microprocessor, a controller, a digital signal processor (DSP), an application specific integrated circuit (ASIC), a field-programmable gate array (FPGA), a video processor, or other equivalent discrete or integrated logic circuitry.

Computer-readable memory 24 is configured to store information and, in some examples, can be described as a computer-readable storage medium. In some examples, a computer-readable memory 24 can include a non-transitory medium. The term “non-transitory” indicates that the storage medium is not embodied in a carrier wave or a propagated signal. In certain examples, a non-transitory storage medium can store data that can, over time, change (e.g., in RAM or cache). In some examples, computer-readable memory 24 can include a temporary memory. As used herein, a temporary memory refers to a memory having a primary purpose that is not long-term storage. Computer-readable memory 24, in some examples, can include volatile memory. As used herein, a volatile memory refers to a memory that does not maintain stored contents when power to the computer-readable memory 24 is turned off. Examples of volatile memories can include random-access memories (RAM), dynamic random-access memories (DRAM), static random-access memories (SRAM), and other forms of volatile memories. In some examples, computer-readable memory 24 is used to store program instructions for execution by processor 22. Computer-readable memory 24, in one example, is used by software or applications running on patient-monitoring system 20 (e.g., by a computer-implemented machine-learning engine or a data-processing module) to temporarily store information during program execution.

Computer-readable memory 24, in some examples, also includes one or more computer-readable storage media. The memory can be configured to store larger amounts of information than volatile memory. The memory can further be configured for long-term storage of information. In some examples, the memory includes non-volatile storage elements. Examples of such non-volatile storage elements can include, for example, magnetic hard discs, optical discs, floppy discs, flash memories, or forms of electrically programmable memories (EPROM) or electrically erasable and programmable (EEPROM) memories.

User interface 26 is an input and/or output device and enables an operator to control operation of patient-monitoring system 20. For example, user interface 26 can be configured to receive inputs from an operator and/or provide outputs related to health condition and/or care for patient 32. User interface 26 can include one or more of a sound card, a video graphics card, a speaker, a display device (such as a liquid crystal display (LCD), a light emitting diode (LED) display, an organic light emitting diode (OLED) display, etc.), a touchscreen, a keyboard, a mouse, a joystick, or other classification of device for facilitating input and/or output of information in a form understandable to users and/or machines.

Video camera 28 is configured to capture video stream 30 of patient 32 and perhaps any other persons who are within its field of view. Video camera 28 is configured to be able to communicate with various software modules and hardware components of patient-monitoring system 20. Video camera 28 can be, for example, a video camera, a webcam, or another suitable source for obtaining video stream 30. In some embodiments, video camera 28 can be controlled by the various software modules executed by processor 22 of patient-monitoring system 20. Video stream 30 can include audiovisual data feeds portraying patient 32. Video stream 30 can be stored to computer-readable memory 24 for use with one or more methods described herein or can be stored to another storage media and recalled to computer-readable memory 24 for use with one or more methods described herein.

Video-Processing Module

Video-processing module 34 includes one or more programs for processing video stream 30, which includes both video data 52 and audio data 54. As used herein, “video data 52” refers to the portion of video stream 30 that is a series of still images and “audio data 54” refers to the sound data stored in video stream 30. Video-processing module 54 can include one or more programs for extracting the video data 52 from the video stream and audio data 54 from the audio data The audio data can include verbal communications and non-verbal sounds. Video-processing module 54 can include one or more programs for extracting semantic text data 56 corresponding to the verbal communications from audio data 54. As used herein, “semantic text data 56” refers to data that represents spoken words, phrases, sentences, and other sounds produced by patient 32 as readable text. Features extracted from video data 52 are features that visually convey information. Features extracted from audio data 54 are features that audibly convey information. Features extracted from semantic text data 56 are features that verbally convey information.

Feature-Extraction Module

Feature-extraction module 36 can include one or more programs for extracting classifiable features from video data 52, audio data 54, and/or semantic text data 56 extracted from video stream 30 by video-processing module 54. Feature-extraction module 36 can classify and/or determine metrics pertaining to the features extracted from video data 52, audio data 54, and/or semantic text data 56. For example, processor 22 can create one or more values that describe the information conveyed by the features extracted from video data 52, audio data 54, and/or semantic text data 56 and store such value or values to computer-readable memory 24 and arrange the values stored in feature sets. First, second, and third feature sets, for example, can correspond to features extracted from video data 52, audio data 54, and semantic text data 56, respectively. In another embodiment, feature sets can include features pertinent to and associated with the various software modules.

Feature extraction from video data 52, audio data 54, and semantic text data 56 can be performed using one or more computer-implemented machine-learning engines that has/have been trained to identify features useful for the specific purposes of the various software modules. The various features identified as useful by the one or more computer-implemented machine-learning engines can then be extracted from video data 52, audio data 54, and semantic text data 56 of video stream 30 of patient 24. The various features identified as being useful for one or more of the software modules can be labeled as such and stored in computer-readable memory, so that the various software modules can identify and retrieve such features used thereby.

The features extracted from video data 52 can include, for example, reference points of the patient's body. Some such reference points can include: forehead location; eye socket locations; chin location; nose location; ear locations; joint locations (e.g., shoulder, elbow, wrist, hip, knee, etc.); head tilt, mouth shape and position; eye shape and position (e.g., eye contact); eyebrow shape and position; etc. Other identified features from video data 52 can include, for example, movements determined using more than one image frame of the video stream. Some such movements can include: hand gestures; head gestures (e.g., nodding or shaking); eye movements (e.g., blinking, looking about, rolling back in the socket, etc.); forehead wrinkling; mouth movements; tongue movements; shoulder movements; body movements (e.g., walking, pacing, sitting up, etc.); among others.

The features extracted from audio data 54 can be, for example, based on vocal pitch, intonation, inflection, sentences stress, or other audio elements that convey information. In at least some examples, the presence of pauses or the absence of speaking in audio data 54 can also convey information. For example, long pauses between words or the absence of speaking may indicate that patient 32 is anxious, bored, and/or distracted, among other options. Portions of audio data 54 that lack vocal-frequency information (e.g., those that correspond to pauses or silence) can be interpreted in combination with features from portions of audio data 54 in which patient 32 is speaking to determine what information, if any, is conveyed by the lack of vocal-frequency information. Portions of audio data 54 that contain vocal-frequency information can include such sounds as: moaning, crying, wailing, stuttering, sighing, yawning, etc.

Semantic text data 56 can be extracted from audio data 54 and/or from video data 52 (e.g., using a lip-reading type of algorithm). Semantic text data 56 refers to data that represents spoken words, phrases, sentences, and other sounds produced by patient 32 as readable text. Semantic text data 56 can be, for example, a transcript of the words spoken in audio data 54. Semantic text data 56 can be extracted from audio data 54 using one or more programs, such as a speech-to-text program. In some examples, video data 52 can be annotated so as to display a text transcript of the words spoken by patient 32. For example, if video data 52 is acquired from a video conferencing platform, the videoconferencing platform may embed a text transcript in video data 52. In these examples, semantic text data 56 can be directly extracted from video data 52 rather than from the extracted audio data 54.

In addition to the above described static features of the feature sets, other features can be determined based on such static features. For example, each of the first, second and third feature sets can include feature combinations of at least two features of a particular feature set or at least two features of the combined feature sets. Such a created features can include metrics related to: a number of times particular feature or a feature combination occurs; a frequency of occurrence of the particular feature of the feature combination; a time period between occurrences of the particular feature or the feature combination; and/or a time period between occurrences of a first feature or a first feature combination and a second feature or a second feature combination, for example.

The features extracted from video data 52, audio data 54, and semantic text data 56 can then be used by the various software modules, which will be described below: to determine a patient's behavior; to assess a confidence level for verbal communications of patient 32; to predict the patient's mental state; to ensemble persons' identities who are present in the patient's room; to monitor the patient's physical environment; to determine the patient's physical location; as well as various other things. Some extracted features can be used to model the patient's movements or lack thereof. Some extracted features can be used to determine a confidence level of a patent's verbal communications. Some extracted features can be used to predict a patient's mental state. Some extracted features can be used to ensemble persons' identities who are captured in video stream 30. Some extracted features can be used to assess a patient's health condition. Some extracted features can be used to predict a patient's health outcome. Some extracted features can be used to enhance care for patient 32. Various examples of these extracted features will be described below with reference to the software modules that utilize them.

Verbal-Confidence Module

Verbal-confidence module 38 is configured to assess a confidence level for the verbal communications of a person. The confidence level for the verbal communications of the person can be assessed using features extracted from video data 52, audio data 54, and/or semantic text data 56. The features used for such assessment of the confidence level for the verbal communications of the person have been identified by a computer-implemented machine-learning engine as being useful for doing such an assessment. The computer-implemented machine-learning engine is trained using a substantial number of video streams of a substantial number of persons making verbal statements that are variously aligned (e.g., some truthful, and others not so) with what is actually believed by these persons, some well aligned and others poorly aligned. The computer-implemented machine-learning engine (e.g., trained as a classification model) scans such video streams for patterns of behavior that are indicative of such a confidence level (e.g., a low or a high confidence level) that the verbal communication is well aligned with the verbal communicator's belief.

Then, the computer-implemented machine-learning engine uses such identified features to create a confidence-assessment model. Verbal-confidence module 38 uses the confidence-assessment model along with the features identified as useful to assess the confidence level for the verbal communications of person. These useful features are extracted from video data 52, audio data 54, and semantic text data 56 and used by the confidence-assessment model to indicate the confidence level for the verbal communications of person.

Various features can be identified by the computer-implemented machine-learning engine as useful for assessing the confidence level for verbal communications of a person. For example, some such features extracted from video data 52 might include, for example: hand gestures, head tilt, the presence and amount of eye contact, the amount of eye blinking, forehead wrinkling, mouth position, mouth shape, eyebrow shape, eyebrow position, eye movements, tongue movements, shoulder movements, pacing, among others. Useful features extracted from audio data 54 can include verbal pitch, stuttering, pauses, nervous tapping, etc. Useful features extracted from semantic text data 56 can include, for example, attestations of truthfulness, such as, for example, “I swear,” “It's true,” “Believe me,” “I wouldn't lie to you,” etc. In addition to such useful features extracted from video data 52, audio data 54, and semantic text data 56, the person's mental state, as predicted using mental-state prediction module 42, can also be used to assess confidence level of the person's verbal communications.

Additional metrics can be developed using such extracted features, such the direction of a gaze of a person, the number of times and/or frequency that a person gazes in a direction, the time period between occurrences of the gaze being directed in a direction, the frequency of the person's hand movements, etc. Verbal-confidence module 38 can use machine learning to identify various feature patterns and feature combinations (a.k.a. “tells”) that are indicative of low and/or high confidence of veracity. The confidence-assessment model, which has been trained using a substantial number of video streams, greatly facilitates finding of such tells that would be nigh impossible in the short timeframe of a typical interaction between a clinician and person, for example. Verbal-confidence module 38 can augment such a general confidence-assessment model, as generated using the substantial number of video streams of a corresponding substantial number of people, with a person-specific confidence-assessment model that is trained using video stream 30 of the person. Such a person-specific confidence-assessment model can improve accuracy of assessing the veracity of verbal communications over assessments of such verbal communications by the general confidence-assessment model alone. Such a person-specific confidence-assessment model improves as the actual veracity or lack thereof of the person's verbal communications becomes known.

Such veracity assessment of verbal communications can be performed in a variety of venues and for a variety of reasons. For example, law enforcement can use such automatic veracity assessment of verbal communications expressed by persons being questioned. The confidence levels of these verbal communications can then be automatically reported to an electronic record and associated with the verbal communications expressed. In another embodiment, healthcare providers can use such automatic veracity assessment of verbal communications expressed by patients for who healthcare is being provided. The confidence levels of these verbal communications can also be automatically reported to an electronic record and associated with the verbal communications expressed.

Patient-Movement Module

Patient-movement module 40 is configured to determine the patient's movements or lack thereof. Patient-movement module 40 can use an anatomic model of a human with various model reference points and/or model reference members. Corresponding patient points and/or patient members of patient 32 can be extracted from video data 52 as human-body features. The patient's reference points and/or reference members can include: forehead location, eye socket locations, chin location, nose location, ear locations, joint locations (e.g., shoulder, elbow, wrist, hip, knee, etc.). From these reference points, the anatomical model corresponding to patient 32 can be sized to his/her stature, and the various members (e.g., humerus, radius, ulna, vertebrae, femur, tibia, fibula, etc.) of the anatomical model can be aligned to correspond with the members of patient 32. From the orientation of the anatomical model, patient-movement module 40 can determine the position of patient 32 (e.g., sitting, lying on back, etc).

Patient-movement module 40 can also use a bed model and/or a room model (e.g., floor surface, walls, door, etc.) to locate the anatomical model of patient 32 with respect to the patient's bed and/or the patient's room. Patient-movement module 40 can then determine the patient's position or movement with respect to the patient's bed and/or the patient's room. Patient-movement module 40 can select from several types of movements, such as, for example: patient 32 lying on back; patient 32 lying on left side; patient 32 lying on right side; patient 32 lying on front; patient 32 moving legs. patient 32 waving arms; patient 32 shaking; patient 32 shivering; patient 32 perspiring; patient 32 sitting up in a bed; patient 32 sitting on a side of the bed; patient 32 getting out of the bed; patient 32 standing; and patient 32 falling; etc. After such patient movements are determined, the patient's movement, as determined by patient-movement module 40, can be communicated to other software modules for use thereby, and/or reported to a digital record and/or medical care personnel.

Mental-State Prediction Module

Mental-state prediction module 42 is configured to predict a mental state of patient 32. The mental state of patient 32 can be predicted using features extracted from video data 52, audio data 54 and/or semantic text data 56. The features used for such prediction of a mental state of patient 32 have been identified by a computer-implemented machine-learning engine as being useful for doing such a prediction. The computer-implemented machine-learning engine is trained using a substantial number of video streams of a substantial number of persons of various mental-states. The computer-implemented machine-learning engine scans such video streams for patterns of behavior that are indicative of the mental states of the persons captured in the video streams.

The computer-implemented machine-learning engine uses such identified features to create a mental-state model. Mental-state prediction module 42 uses the mental-state model along with the features identified as useful to predict a mental state of patient 32. The features extracted from video data 52, audio data 54, and semantic text data 56 are used by the mental-state model to predict the mental state of patient 32.

Various features might be identified by the computer-implemented machine-learning engine as useful for predicting the mental state of patient 32. For example, some such features extracted from video data 52 can include, for example: hand gestures, head tilt, the presence and amount of eye contact, the amount of eye blinking, forehead wrinkling, mouth position, mouth shape, eyebrow shape, eyebrow position, eye movements, tongue movements, shoulder movements, pacing, among others. Useful features extracted from audio data 54 can include verbal pitch, stuttering, pauses, nervous tapping, wailing, crying, moaning, etc. Useful features extracted from semantic text data 56 can include, for example, statements regarding feelings or condition, such as, for example, “I'm sad,” “Ouch,” “It's painful,” “I wish it were over,” etc.

In some embodiments, the mental-state model is a multidimensional mental-state model. Such a multidimensional mental-state model can include a plurality of dimensions, each of which can correspond to a different aspect of mental state. Such multidimensional mental-state models can describe mental state more accurately than existing models of mental state. Because mental-state models can more accurately describe a patient's mental state, multidimensional mental-state models can significantly improve the resolution and accuracy of predictions of mental state as compared to existing models, including single-dimensional models of mental state.

In one embodiment, the multidimensional mental-state model has a first dimension and a second dimension. The first dimension can represent an intensity of a patient's mental state and the second dimension can represent a pleasantness of the patient's mental state, for example. Different mental states can be described by different combinations of values in the first dimension and second dimension. For example, each quadrant of multidimensional mental-state model can represent a different mental state or different subregions (including subregions entirely within and/or extending across quadrants of the multidimensional mental-state model) of the multidimensional mental-state model can represent different mental states.

Additionally or alternatively, the dimensions of multidimensional mental-state model can represent a mental state by describing aspects of information communicated by patient 32 (i.e., in video data 52, audio data 54, and/or semantic text data 56 for a patient), such as the relative importance of the information patient 32 is conveying information, the positivity of the information patient 32 is conveying, and/or the subject of the conversation in which patient 32 is participating (e.g., whether the subject is administrative, technical, etc.), among other options. The importance of the information that patient 32 is conveying can be assessed based on, for example, a task or job that patient 32 is performing.

In other examples, each of the first dimension and the second dimension can represent separate and/or distinct mental states. For example, the first dimension can represent a first mental state, such as confusion, and the second dimension can represent a second mental state, such as calmness. Various regions, such as quadrants, of the multidimensional mental-state model can represent different combinations of confusion and calmness, with each region representing a discrete overall mental state. Simultaneously monitoring confusion and calmness can facilitate, for example, a measurement of how well patient 32 and a visitor are understanding one another. Specifically, a quadrant with positive confusion and calmness values can represent an overall “confused and attentive” mental state; a quadrant with negative confusion and positive calmness values can represent an overall “comprehending and attentive” mental state; a quadrant with negative confusion and negative calmness can represent an overall “comprehending and inattentive” mental state; and a quadrant with positive confusion and negative calmness can represent an overall “confused and inattentive” mental state.

In other examples, the dimensions of the multidimensional mental-state model can represent any other combination of mental states. For example, the dimensions of multidimensional mental-state model can also include one or more of tiredness, sleepiness, serenity, satisfaction, calmness, relaxation, contentment, distress, frustration, anger, annoyance, tension, fear, alarm, misery, sadness, depression, gloom, boredom, astonishment, amusement, excitement, happiness, delight, gladness, pleasure, thankfulness, gratitude, confusion, smugness, deliberation, anticipation, cheer, sympathy, trust, humor, envy, melancholy, hostility, resentment, revulsion, and/or ennui. As a specific example, the multidimensional mental-state model can include three dimensions, where each dimension represents an intensity of a specific mental state. The three dimensions can represent intensities of, for example, frustration, fear, and excitement, respectively. Metrics for each dimension can be generated by a computer-implemented machine-learning model corresponding to each dimension.

Different ordered combinations of metrics from the first and second dimension can represent different combinations of values along the first dimension and the second dimension of the multidimensional mental-state model. In examples where the first dimension and the second dimension represent intensity and pleasantness of a patient's mental state, respectively, an ordered combination of metrics might correspond to a mental state having relatively high intensity and relatively high pleasantness, such as happiness. The second ordered combination of metrics might correspond to a mental state having relatively high intensity and relatively low pleasantness, such as frustration or annoyance. The third ordered combination of metrics might correspond to a mental state having low intensity and low pleasantness, such as boredom. The fourth ordered combination of metrics might correspond to a mental state having low intensity and high pleasantness, such as relaxation.

As will be explained in further detail subsequently, multidimensional mental-state models can more accurately describe the mental state of a patient than mental-state models having only a single dimension. For example, the multidimensional mental-state model enables the mental states of amusement, excitement, happiness, delight, gladness, and pleasure to be distinguished. Existing, one-dimensional models of mental state are unable to clearly distinguish between closely related mental states. Further, multidimensional mental-state models having more than two dimensions can more accurately describe the mental state of a patient than mental-state models having only two dimensions. For example, it is possible for a patient to be confused, envious, and sleepy simultaneously. A three-dimensional mental-state model having dimensions describing each of confusion, envy, and sleepiness can more accurately describe the mental state of a patient experiencing all three mental states to varying degrees than existing representations or models of mental state. As such, the use of a multidimensional mental-state model enables significantly more accurate prediction of a patient's mental state.

Mental-state prediction module 42 can be used to generate metrics for each dimension of the multidimensional mental-state model. In some examples, mental-state prediction module 42 can extract features from image data 52, audio data 54, and semantic text data 56 to generate metrics for each of the first dimension and the second dimension (of a two-dimensional model). The use of different combinations of the features sets (e.g., combining features extracted from two or three of image data 52, audio data 54, and semantic text data 56) provide further advantages and improvements to both the efficiency and accuracy of mental-state models that do not provide such combinations of features. More specifically, excluding different combinations of image, audio, and text data permits mental state predictions to be made using only predictive data rather than non-predictive data. For example, semantic text data 56 may offer significantly more insight into the importance of a particular discussion than image data 52 or audio data 54 alone. The multidimensional mental-state model can be configured so that only features from semantic text data 56 are used to calculate the dimension associated with discussion importance, which can improve accuracy of the predicted patient's mental state by disregarding non-predictive data and, consequently, improving efficiency by only requiring one classification of data (i.e., semantic text data) to calculate the dimensional value for the discussion importance dimension.

While a multidimensional mental-state model can have as few as two dimensions—first dimension and second dimension, additional dimensions can be added to multidimensional mental-state model as required for a given application and/or operational need. Adding additional dimensions to multidimensional mental-state model can distinguish between mental states that are nearby or similar to one another, thereby improving the resolution of multidimensional mental-state model. For example, additional dimensions describing information importance, information positivity, the subject of the information (i.e., whether the information is administrative, technical, etc.), and/or other mental states can further be used to resolve and distinguish between similar overall mental states. In examples where each dimension of the multidimensional mental-state model represents a separate mental state (e.g., one or more of confusion, envy, calmness, sleepiness, etc.), the inclusion of additional dimensions can also provide more accurate description of a patient's mental state.

In operation, mental state prediction module 52 facilitates the prediction of the patient's mental state based only on information communicated by patient 32 in video stream 30 captured by video camera 28. Conventional methods of predicting mental state rely on complex biometric data. Collecting biometric data can require complex machines and, further, often requires physically-intrusive methods. Conversely, mental state prediction module 42 predicts the patient's mental state using only video stream 30, which can be collected using only video camera 28 and without the use of any physically-intrusive techniques.

Identity-Ensembling Module

Identity-ensembling module 44 is configured to ensemble the identify of persons captured in video stream 30. Identification of persons captured in video stream 30 can be ensembled using features extracted from video data 52, audio data 54, and/or semantic text data 56. The features used for such identification of persons captured in the video stream have been identified by a computer-implemented machine-learning engine as being useful for doing such identity ensembling. The computer-implemented machine-learning engine is trained using a substantial number of video streams of a substantial number of different persons. The computer-implemented machine-learning engine scans such video streams for personal attributes that distinguish persons one from another. Data-bases of such persons and their distinguishing attributes are maintained by various entities that perform such identification of persons.

The computer-implemented machine-learning engine uses such identified features to create a computer-implemented identity-ensembling model. Various features can be identified by the computer-implemented machine-learning engine as useful for ensembling the identities of persons captured in the video stream. For example, features extracted from video data 52 can include distinguishing visual attributes of persons, such as, for example, relative location of facial features, color of hair and eyes, etc. Useful features extracted from audio data 54 can include spectral frequency of voice, cadence of verbal communications, audio level of voice, etc. Useful features extracted from semantic text data 56 can include turns of phrase, vocabulary, accent, etc. Identity-ensembling module 44 can use the mental-state model in addition to the features identified as useful for identity ensembling. The features extracted from video data 52, audio data 54, and semantic text data 56 (as well as the predicted mental states of persons, if used in the model) are used by the computer-implemented identity-ensembling model to ensemble the identities of the persons captured in the video stream.

The identities of persons captured in the video stream can be ensembled by, for example, cross-referencing features extracted from video data 52, audio data 54, and semantic text data 56 with a table, array, or other data structure that relates features from video, audio, and/or text data to identity. In other examples, identities can be ensembled using the identity-ensembling model trained to identify persons based on a training set of features from video, audio, and/or semantic text data 56. In these examples, the identity ensembled can include, for example, descriptions of the name, title, or organizational position of the person, among other options. Additionally, and/or alternatively, the identity can include descriptions of the physical appearance, setting, built environment, or geographic location of the person, among other options.

Using such ensembled identities, patient interactions can be classified. For example, interactions with persons identified as visitors can be distinguished from interactions with healthcare workers. Metrics regarding number, time duration, and frequencies of various types of patient interactions can be generated. Such metrics can be stored as features and then used by the computer-implemented machine-learning engines for use with the various software modules.

Behavior-Alerting Module

Behavior-alerting module 46 is configured to automatically invoke an alert if the patient's behavior is considered to be an alerting behavior. Patient behaviors can include the patient's physical movement or lack thereof as determined by patient movement module 40, the patient's mental state as predicted by mental-state prediction module 52, verbal communications as extracted from semantic text data 56, and non-verbal sounds as extracted from audio data 54. The patient's behavior is considered to be an alerting behavior if it is found in a set of alerting behaviors corresponding to a patient's classification of patient 32 being captured in the video stream.

Cataloging behaviors into sets of alerting behaviors for the various patient classifications can be performed in various manners. In one embodiment, for example, behaviors are identified as alerting behaviors for the various patient classifications by an expert. Such an expert can catalogue sets of alerting behaviors corresponding to patient classifications and patient attributes. Rules can be established so as to draw from these sets the alerting behaviors corresponding to each specific patient. Such sets of alerting behaviors can be stored in computer-readable memory 24 for use with behavior-alerting module 46. In another embodiment, at least some behaviors can be designated as alerting behaviors for certain patient classifications using a computer-implemented machine-learning engine trained using a substantial number of video streams of a substantial number of patients behaving in various manners. The computer-implemented machine-learning engine scans such video streams for behaviors that contributed to deleterious consequences to patient 32. In another embodiment the behaviors identified as alerting behaviors for the various patient classifications can be listed by an expert. Such lists can be stored in computer-readable memory 24 for use with behavior-alerting module 46. In still another embodiment, the set of alerting behaviors can be designated by the medical care provider, such as, for example, the patient's doctor(s).

### Example System for Automatically Generating an Alert

Obtaining metrics of patient behavior using a video stream or an audio stream can be used for a variety of purposes. One such example will be described with reference to FIGS. 2A and 2B. FIG. 2A is a perspective view of a patient's room, in which a patient-monitoring system 20 is monitoring the patient's behavior. In FIG. 2A, patient 32 is lying down in hospital bed 58 while being monitored by video camera 28 located within patient's room 60. Video camera 28 is providing video stream 30 to behavior-alerting module 46 (depicted in FIG. 1). The patient-monitoring system 20 includes a processor and computer readable memory, which has been encoded with instructions that, when executed by the processor, cause behavior-alerting module 46 to invoke an alert if the patient's behavior, as determined by patient behavior module 40, is determined to merit such an alert. The patient's behavior merits such an alert if the patient's behavior, as determined by patient-behavior module 40, is found in a set 72 of alerting behaviors 72a-72n corresponding to the patient's classification of patient 32.

In the embodiment depicted in FIGS. 2A and 2B, patient 32 is a sedentary patient that has difficulty moving. Such sedentary patients, as patient 32, are at risk of developing pressure ulcers. Pressure ulcers are fairly common ailments that sedentary patients can develop during hospitalization. Pressure ulcers are also largely preventable ailments. Because pressure ulcers are preventable, a hospital can be held responsible for costs associated with treating pressure ulcers that developed while patient 32 is being treated therewithin. When a pressure ulcer develops, the hospital can be required to publicly report the ailment to a government agency. Such a report can tarnish the public reputation pertaining to the hospital. Therefore, preventing pressure ulcers, for example, can limit costs incurred by the hospital, help the hospital to maintain its good reputation, as well as enhance the care provided to patient 32.

One relatively effective way to inhibit development of pressure ulcers is to physically move (e.g., turn) patients who have been in a sedentary position for a predetermined period of time. Thus, by monitoring patient 32, such sedentary behaviors can be determined and timed. Should patient 32 remain in such a sedentary position longer than the predetermined period of time, an alert can be invoked so as to inform nurse 62 at nursing station 64 that patient 32 should be turned.

To accomplish such an alerting invocation, behavior-alerting module 46 causes the patient-monitoring system 20 to extract video data 52, audio data 54, and semantic text data 56 from video stream 30 of patient 32. Patient-behavior module 46 causes the patient-monitoring system 20 to analyze video data 52 to identify first feature set 66 of video features identified by a computer-implemented machine-learning engine as being indicative of at least one of a set 72 of alerting behaviors 72a-72n corresponding to a patient classification of the patient. Patient-behavior module 46 causes the patient-monitoring system 20 to analyze audio data 54 to identify second feature set 68 of audio features identified by the computer-implemented machine-learning engine as being indicative of at least one of the set 72 of alerting behaviors 72a-72n. Patient-behavior module 46 causes the patient-monitoring system 20 to analyze semantic text data 56 to identify third feature set 70 of semantic text features identified by the computer-implemented machine-learning engine as being indicative of at least one alerting behavior of the set 72 of alerting behaviors 72a-72n. First feature set 66 includes features that visually indicate information. Second feature set 68 includes features that audibly convey information. Third feature set 70 includes features that verbally convey information. Each of these three feature sets 66, 68, and 70 can be used alone or in combination with the other feature sets to generate metrics pertaining to: i) a patient's movement or lack thereof; ii) a patient's mental state, iii) a patient's non-verbal sounds; iv) a patient's verbal communications; v); confidence levels of a patient's verbal communications; vi) presence and/or identities of a patient's visitors and interactions of patient 32 therewith; and vii) environmental conditions of the patient's room, etc. For example, alerting behaviors pertaining to the patient's movement or lack thereof.

Patient-behavior module 40 then causes the patient-monitoring system 20 to determine the patient's behavior 68 of patient 32 based on first feature set 66, second feature set 68, and/or third feature set 70. The patient's behavior 68 is determined using computer-implemented machine-learning model (e.g., a patient-behavior prediction model) generated by the computer-implemented machine-learning engine. Behavior-alerting module 46 causes the patient-monitoring system 20 to compare the determined patient behavior 86 with a set 72 of alerting behaviors 72a-72n corresponding to a patient classification of patient 32. Patient 32 of FIG. 2A has been classified, at least in part, as a sedentary patient. Therefore, one alerting behavior 72a corresponding to such a sedentary patient classification is patient 32 lying in one position for a threshold period of time, as determined by patient-behavior module 40. Such sedentary stillness is one such physical movement or lack thereof of a category of patient behaviors that can be determined by patient-behavior module 40. Such physical movements or lack thereof, as determined by patient-behavior module 40 can include, for example: patient 32 lying on patient 32's back; patient 32 lying on the patient's left side; patient 32 lying on the patient's right side; patient 32 lying on the patient's front; patient 32 moving the patient's legs; patient 32 waving the patient's arms; patient 32 shaking; patient 32 shivering; patient 32 perspiring; patient 32 sitting up in a bed; patient 32 sitting on a side of the bed; patient 32 getting out of the bed; patient 32 standing; and patient 32 falling; etc. Patient-behavior module 40 can also determine other metrics related to these physical movements or lack thereof, such as, for example, frequency of such movements, time duration of such movements, time of day, time between movements, etc.

Behaviors other than physical behaviors can also be considered to be alerting behaviors for various classifications of patients. For example, mental states of the patient can be included in the set of alerting behaviors. Verbal statements can be included in the set of alerting behaviors. For example, the following categories of verbal statements can be alerting behaviors: a request for assistance; an expressed lament; an expressed concern; an expression of worry; an expression of sorrow; a statement of pain, etc. Non-textual sounds made by the patient can be included in the set of alerting behaviors. For example, the following categories of non-textual sounds can be alerting behaviors: crying; groaning; moaning; whimpering; sounds of breathing difficulty, etc.

Behavior-alerting module 46 causes the patient-monitoring system 20 to compare the patient's behaviors, as determined by behavior-alerting module 46, with the set 72 of alerting behaviors 72a-72n. As indicated above, one such alerting behavior 72a is lying in one position for a predetermined length of time. When behavior-alerting module 46 determines that patient 32 has remained in one such lying position for the predetermined period of time, behavior-alerting module 46 causes the patient-monitoring system 20 to automatically invoke an alert. When such an alert is invoked, nurse 62 at nursing station 74 is alerted to the sedentary condition of patient 32. Nurse 62 can then enter the room of patient 32 and turn patient 32 so as to lie in a different position (e.g., turn patient 32 from back to side). Such turning of patient 32 can prevent pressure ulcers from developing.

Although sometimes it is described that the software modules, such as the behavior-alerting module, perform one or more operation, such modules typically are coded instructions that cause the system to perform the operations described.

Assessing a Confidence Level of Verbal Communications

FIG. 3 is a flowchart for a method for assessing a confidence level of verbal statements expressed by a person during a verbal communication. The method depicted in FIG. 3 can be encoded as program instructions, which are then stored in computer-readable memory 24 and executed by processor 22 (depicted in FIG. 2B). In FIG. 3, the method 80 begins at step 82, where processor 22 receives video stream 30 of the verbal communication (e.g., a verbal communication involving patient 32 in patient room 60 as depicted in FIG. 2A). At step 84, processor 22 extracts video data 52, audio data 54, and semantic text data 56 from video stream 30 of the verbal communication. At step 86, processor 22 analyzes video data 52 to identify first feature set 66 of video features identified by a computer-implemented machine-learning engine as being indicative of veracity of verbal statements expressed by the person. At step 88, processor 22 analyzes audio data 54 to identify second feature set 68 of audio features identified by the computer-implemented machine-learning engine as being indicative of veracity of verbal statements expressed by the person. At step 90, processor 22 analyzes semantic text data 56 to identify third feature set 70 of semantic text features identified by the computer-implemented machine-learning engine as being indicative of veracity of verbal statements expressed by the person. Then, at step 92, processor 22 determines additional confidence-assessment metrics of verbal communications of patient 32 based on combinations of features identified in first, second, and/or third features sets 66, 68, and/or 70. Such additional confidence-assessment metrics are determined using a computer-implemented machine-learning engine. These additional confidence-assessment metrics can be included in first, second, and/or third feature sets 66, 68, and/or 70 or as a fourth feature set, which can involve combinations of features from the first, second, and/or third sets 66, 68, and/or 70. At step 94, processor 22 assesses the confidence levels of the verbal statements of the person based on the features and/or metrics of these feature sets. The confidence levels are assessed using a computer-implemented machine-learning model generated by the computer-implemented machine-learning engine. At step 96, processor 22 associates the confidence levels with the verbal statements to which the confidence levels pertain. Then, at step 98, processor 22 reports the confidence levels of the verbal statements expressed by the person. Such reporting can be performed in a variety of ways. For example, a text log of the verbal communications can be annotated with such a confidence level at the location therewithin corresponding to the associated time of the video stream. In another embodiment, the confidence level and the associated time of the video stream can be transmitted to a healthcare professional, such as, for example, to a doctor or to a nurse.

In some embodiments, instead of associating the confidence levels with the verbal statements, the confidence levels assessed are associated with the feature or feature combinations from which the confidence levels are derived. In such embodiments, these confidence levels can be compared with a confidence level threshold. When an assessed confidence level drops below the confidence level threshold, a low-confidence indication can be reported. Such a low-confidence level indication, for example, can be annotated in the video stream. In some embodiments, the feature or feature combination from which the low-confidence level was derived can be identified (e.g., in the video stream).

The computer-implemented machine-learning model that assesses confidence levels of verbal communications is a product of training of the computer-implemented machine-learning engine. The computer-implemented machine-learning engine is trained using a plurality of training video streams of a corresponding plurality of training persons. Training video data, training audio data, and training semantic text is extracted from a plurality of training video streams of the corresponding plurality of training persons. The extracted training video data are analyzed to identify a first training feature set of video features. The extracted training audio data are analyzed to identify a second training feature set of audio features. The extracted training semantic text data are analyzed to identify a third training feature set of semantic text features. A plurality of known confidence levels corresponding to the verbal statements of the plurality of training person captured in the plurality of training video streams is received in some fashion by the computer-implemented machine-learning engine. General model coefficients of the computer-implemented machine-learning model are determined so as to improve a correlation between the plurality of known training confidence levels and a plurality of confidence levels as determined by the computer-implemented machine-learning model. In some embodiments, model features from the first, second, and third training feature sets are selected as being indicative of the known confidence levels corresponding to the verbal statements of the plurality of training persons captured in the plurality of training video streams. Sometimes some of the first, second, and third training feature sets may not be very indicative of the known confidence levels corresponding to the verbal statements of the plurality of training persons captured in the plurality of training video streams. Such not-very-indicative features are sometimes not selected for use in the computer-implemented machine-learning model.

In some embodiments, the video stream of the person can be added to the plurality of training videos along with the known veracities of verbal communications of the person. Such addition of the video stream of the person can augment the plurality of the training video streams if the veracities of some of the verbal statements of the person are known. In some embodiments, the computer-implemented machine-learning model that is generated as described above is a general veracity-assessment model. Such a general veracity-assessment model can be augmented by a person-specific veracity-assessment model. Training of the person-specific behavior model can be performed in a manner similar to the training of the general person-behavior model. A set of veracity-known video verbal statements expressed by the person can be identified. Each of the veracity-known verbal statements are assigned a known confidence level. Person-specific video data, person-specific audio data, and person-specific semantic text data are extracted from the set of video stream capturing the veracity-known verbal statements expressed by the person. The person-specific video data are analyzed to identify a first person-specific feature set of video features. The person-specific audio data are analyzed to identify a second person-specific feature set of audio features. The person-specific semantic text data are analyzed to identify a third person-specific feature set of semantic text features. Then, person-specific model coefficients of a person-specific veracity-assessment model are determined. Such person-specific model coefficients are determined so as to improve a correlation between the known confidence levels and confidence levels as determined by the person-specific veracity-assessment model.

Automatically Invoking an Alert

FIG. 4 is a flowchart of a method for automatically invoking an alert when a patient's behavior is an alerting behavior. The method depicted in FIG. 4 can be encoded as program instructions, which are then stored in computer-readable memory 24 and executed by processor 22 (depicted in FIG. 2B). In FIG. 4, the method 100 begins at step 102, where processor 22 receives video stream of patient 32 in patient room 60 (depicted in FIG. 2A). At step 104, processor 22 extracts video data 52, audio data 54, and semantic text data 56 from video stream 30 of patient 32. At step 106, processor 22 analyzes video data 52 to identify first feature set 66 of video features identified by a computer-implemented machine-learning engine as being indicative of at least one of a set of alerting behaviors corresponding to a patient classification of patient 32. At step 108, processor 22 analyzes audio data 54 to identify second feature set 68 of audio features identified by the computer-implemented machine-learning engine as being indicative of at least one of the set of alerting behaviors. At step 110, processor 22 analyzes semantic text data 56 to identify third feature set 70 of semantic text features identified by the computer-implemented machine-learning engine as being indicative of at least one of the set of alerting behaviors. Then, at step 112, processor 22 determines patient behavior 36 of patient 32 based on first, second, and/or third features sets 66, 68, and/or 70. Patient behavior 36 is determined using a computer-implemented machine-learning model generated by the computer-implemented machine-learning engine. At step 114, processor 22 compares patient behavior 36 with the set of alerting behaviors. If, at step 114, patient behavior 36 is found in the set of alerting behaviors, then method 100 advances to step 116, where processor 22 automatically invokes the alert when patient behavior 36 is determined to be included in the set of alerting behaviors. If, however, at step 114, patient behavior 36 is not found in the set of alerting behaviors, then method 100 returns to step 104 and continues extracting video data 52, audio data 54, and semantic text data 56 from the video stream of patient 32.

The computer-implemented machine-learning engine that determines the patient's behavior is a product of training the computer-implemented machine-learning engine. Training of the computer-implemented machine-learning model uses a plurality of training video streams of a corresponding plurality of training patients. Training video data, training audio data, and training semantic text is extracted from a plurality of training video streams of the corresponding plurality of training patients. The extracted training video data are analyzed to identify a first training feature set of video features. The extracted training audio data are analyzed to identify a second training feature set of audio features. The extracted training semantic text data are analyzed to identify a third training feature set of semantic text features. A plurality of known training patient behaviors corresponding to of the plurality of training patients captured in the plurality of training video streams is received in some fashion by the computer-implemented machine-learning engine. General model coefficients of the computer-implemented machine-learning model are determined so as to improve a correlation between the plurality of known training patient behaviors and a plurality of training patient behaviors as determined by the computer-implemented machine-learning model. In some embodiments, model features from the first, second, and third training feature sets are selected as being indicative of the known patient behaviors corresponding to of the plurality of the training patients captured in the plurality of the training video streams. Sometimes some of the first, second, and third training feature sets are selected may not be very indicative of the known patient behaviors corresponding to of the plurality of the training patients captured in the plurality of the training video streams. Such not-very-indicative features are sometimes not selected for use in the computer-implemented machine-learning model.

In some embodiments, the video stream of the patient can be added to the plurality of training videos along with the known patient behaviors of the patient. Such addition of the video stream of the patient can augment the plurality of the training video streams if the some of the behaviors of the patient are known. In some embodiments, the computer-implemented machine-learning model is a general patient-behavior model, which can be augmented by a patient-specific behavior model. Training of the patient-specific behavior model can be performed in a manner similar to the training of the general patient-behavior model. A set of behavior-known video portions of the patient are identified. Each of the behavior-known video stream portions capture features indicative of known patient-specific behaviors. Patient-specific video data, patient-specific audio data, and patient-specific semantic text data are extracted from the set of behavior-known video portions of the patient. The patient-specific video data are analyzed to identify a first patient-specific feature set of video features. The patient-specific audio data are analyzed to identify a second patient-specific feature set of audio features. The patient-specific semantic text data are analyzed to identify a third patient-specific feature set of semantic text features. The known patient-specific behaviors corresponding to the patient captured in the video stream are received is some fashion. Then, patient-specific model coefficients of a patient-specific patient-behavior model are determined. Such patient-specific model coefficients are determined so as to improve a correlation between the known patient-specific behaviors and patient behaviors as determined by the patient-specific patient-behavior model.

Behavior-Alerting Module Using Only Audio Data

In some embodiments, the patient's room will not be equipped with a video camera. In such situations, many of the behaviors determined using video data 52, audio data 54, and semantic text data 56, can be determined using only audio data 54 and/or semantic text data 56.

FIG. 5 is a flowchart of a method for automatically invoking an alert when a patient's behavior, as determined by audio data 54 and semantic text data 56, is an alerting behavior. The method depicted in FIG. 5 can be encoded as program instructions, which are then stored in computer-readable memory 24 and executed by processor 22 (depicted in FIG. 2B). In FIG. 5, the method 120 begins at step 122, where processor 22 receives an audio stream of sounds within patient room 60 (depicted in FIG. 2A). At step 124, processor 22 extracts audio data 54 and semantic text data 56 from the audio stream. At step 126, processor 22 analyzes audio data 54 to identify first feature set 32 of audio features identified by the computer-implemented machine-learning engine as being indicative of at least one of the set of alerting behaviors corresponding to a patient classification of patient 32. At step 128, processor 22 analyzes semantic text data 56 to identify second feature set 34 of semantic text features identified by the computer-implemented machine-learning engine as being indicative of at least one of the set of alerting behaviors. Then, at step 130, processor 22 determines patient behavior 36 of patient 32 based on first and/or second features sets 32 and/or 34. Patient behavior 36 is determined using a computer-implemented machine-learning model generated by the computer-implemented machine-learning engine. At step 132, processor 22 compares patient behavior 36 with the set of alerting behaviors. If, at step 132, patient behavior 36 is found in the set of alerting behaviors, then method 120 advances to step 134, where processor 22 automatically invokes the alert when patient behavior 36 is determined to be included in the set of alerting behaviors. If, however, at step 132, patient behavior 36 is not found in the set of alerting behaviors, then method 120 returns to step 124 and continues extracting audio data 54 and semantic text data 56 from the video stream of patient 32.

Health-Outcome Prediction Module

Health-outcome prediction module 48 is configured to predict a health outcome of patient 32 captured in the video stream. Traditionally, doctors relied primarily on the traditional health data of patient 32 (e.g., diagnosed condition, heart rate, body temperature, respiratory rate, arterial blood pressures, lab results, medications administered, etc.) to predict the health outcome of patients. Other metrics obtained from the video stream can be used to augment the health data to provide a more comprehensive set of metrics for use in predicting the health outcome of patient 32. Patient behaviors, including the mental state of patient 32, patient interactions, patient room environment, can be such additional metrics used in predicting a health outcome of patient 32.

The health outcome of patient 32 captured in the video stream can be predicted using metrics generated from the features extracted from video data 52, audio data 54 and/or semantic text data 56. The metrics used for such prediction of the health outcome of patient 32 captured in the video stream can be identified by a computer-implemented machine-learning engine as being useful for doing such prediction. The computer-implemented machine-learning engine is trained using a substantial number of video streams of a substantial number of different patients. The computer-implemented machine-learning engine scans such video streams for metrics that distinguish health outcomes for patients of various patient classifications.

Then, the computer-implemented machine-learning engine uses such identified metrics to create a health-outcome model. Health-outcome prediction module 48 uses the mental-state model along with the metrics identified as useful for health-outcome prediction. The metrics generated from the features extracted from video data 52, audio data 54, and semantic text data 56 are used by the health-outcome model to predict the health outcome of patient 32 captured in the video stream.

FIG. 6 is a flowchart of a method for predicting a health outcome for a video-monitored patient. The method depicted in FIG. 6 can be encoded as program instructions, which are then stored in computer-readable memory 24 and executed by processor 22 (depicted in FIG. 2B). In FIG. 6, the method 140 begins at step 142, where processor 22 receives video stream of patient 32 in patient room 60 (depicted in FIG. 2A). At step 144, processor 22 extracts video data 52, audio data 54, and semantic text data 56 from a video stream of patient 32. At step 146, processor 22 analyzes video data 52 to identify first feature set 66 of video features identified by a computer-implemented machine-learning engine as being indicative of at least one of a set of health outcomes corresponding to a patient classification of patient 32. At step 148, processor 22 analyzes audio data 54 to identify second feature set 68 of audio features identified by the computer-implemented machine-learning engine as being indicative of at least one of the set of health outcomes corresponding to the patient's classification of patient 32. At step 150, processor 22 analyzes semantic text data 56 to identify third feature set 70 of semantic text features identified by the computer-implemented machine-learning engine as being indicative of at least one of the set of health outcome corresponding to the patient's classification of patient 32. Then, at step 152, processor 22 predicts the health outcome of patient 32 based on first, second and/or third features sets 30, 32 and/or 34. The health outcome is predicted using a computer-implemented machine-learning model generated by the computer-implemented machine-learning engine. At step 154, processor 22 reports the health outcome predicted.

Reporting the predicted health outcome can take different forms in different embodiments. For example, in one embodiment, the predicted health outcome of the patient can be automatically reported in a digital medical record associated with the patient. In some embodiments, the predicted health outcome of the patient can be automatically reported to a medical care facility in which the patient is being cared. In some embodiments, the predicted health outcome of the patient can be automatically reported to a doctor caring for the patient. In some embodiments, multiple reporting avenues can be performed, such as, for example, any combination of the above disclosed reporting avenues.

The computer-implemented machine-learning engine that predicts a healthcare outcome of a patient is a product of training the computer-implemented machine-learning engine. Training of the computer-implemented machine-learning model uses a plurality of training video streams of a corresponding plurality of training patients. Training video data, training audio data, and training semantic text is extracted from a plurality of training video streams of the corresponding plurality of training patients. The extracted training video data are analyzed to identify a first training feature set of video features. The extracted training audio data are analyzed to identify a second training feature set of audio features. The extracted training semantic text data are analyzed to identify a third training feature set of semantic text features. A plurality of known training health outcomes corresponding to each of the plurality of training patients captured in the plurality of training video streams is received in some fashion by the computer-implemented machine-learning engine. General model coefficients of the computer-implemented machine-learning model are determined so as to improve a correlation between a plurality of known training health outcomes and a plurality of training patient health outcomes as determined by the computer-implemented machine-learning model. In some embodiments, model features from the first, second, and third training feature sets are selected as being indicative of the known training healthcare outcomes corresponding to the plurality of training patients captured in the plurality of training video streams. Sometimes some of the first, second, and third training feature sets are selected may not be very indicative of the known healthcare outcomes corresponding to of the plurality of the training patients captured in the plurality of the training video streams. Such not-very-indicative features are sometimes not selected for use in the computer-implemented machine-learning model.

Care-Enhancement Module

Care-enhancement module 50 is configured to enhance care for patient 32 captured in the video stream. Such care for patients can be enhanced using machine learning of health outcomes for a substantial number of patients captured in a corresponding substantial number of video streams. The computer-implemented machine-learning engine used to predict health outcomes for specific patients has been trained using video streams of patients whose health outcomes are known. Therefore, the computer-implemented machine-learning engine can be trained to identify metrics based on the features extracted from video data 52, audio data 54 and/or semantic text data 56. The metrics used for such enhancement of care for patient 32 captured in the video stream can include those identified by the computer-implemented machine-learning engine as being useful for predicting health outcome. The computer-implemented machine-learning engine scans such video streams for metrics that distinguish health outcomes for patients of various patient classifications.

Then, the computer-implemented machine-learning engine uses such identified metrics to create a care-enhancement model. Care-enhancement module 50 uses the care-enhancement model along with the metrics identified as useful for care-enhancement for patient 32. The metrics generated from the features extracted from video data 52, audio data 54, and semantic text data 56 are used by the care-enhancement model to make recommendations to the healthcare professionals for enhancing care patient 32 captured in the video stream.

FIG. 7 is a flowchart of a method for recommending changes in the care for a video-monitored patient. The method depicted in FIG. 7 can be encoded as program instructions, which are then stored in computer-readable memory 24 and executed by processor 22 (depicted in FIG. 2B). In FIG. 7, the method 170 begins at step 172, where processor 22 receives video stream of patient 32 in patient room 60 (depicted in FIG. 2A). At step 174, processor 22 extracts video data 52, audio data 54, and semantic text data 56 from a video stream of patient 32. At step 176, processor 22 analyzes video data 52 to identify first feature set 66 of video features identified by a computer-implemented machine-learning engine as being indicative of at least one of a set of health outcomes of training patients classified with a patient classification of patient 32. At step 178, processor 22 analyzes audio data 54 to identify second feature set 68 of audio features identified by the computer-implemented machine-learning engine as being indicative of at least one of the set of health outcomes of the training patients classified with the patient's classification of patient 32. At step 180, processor 22 analyzes semantic text data 56 to identify third feature set 70 of semantic text features identified by the computer-implemented machine-learning engine as being indicative of set of at least one of the set of health outcomes of the training patients classified with the patient's classification of patient 32. Then, at step 182, processor 22 predicts the health outcome of patient 32 based on the first, second, and/or third features sets 66, 68, and/or 70. The health outcome of patient 32 is predicted using a computer-implemented machine-learning model generated by the computer-implemented machine-learning engine. At step 184, processor 22 compares the health outcome predicted with the set of health outcomes of the training patients classified with the patient's classification of patient 32. Video streams of the training patients were used to train the computer-implemented machine-learning engine in generating the computer-implemented machine-learning model. At step 186, processor 22 identifies differences between first, second, and/or third feature sets 30, 32 and/or 34 corresponding to patient 32 and feature sets of the training patients who have better health outcomes than the health outcome predicted for patient 32. At step 18, processor 22 reports the differences identified.

In some embodiments, features that are common to the feature sets of the training patients who have better health outcomes than the predicted health outcome of the patient are identified. Such features can then be automatically reported to the healthcare provider. In some embodiments, the identified features and/or the predicted healthcare outcome can be automatic reported to a digital medical record associated with the patient. In some embodiments, the identified features and/or the predicted healthcare outcome can be automatic reported to the medical doctor who is caring for the patient.

The computer-implemented machine-learning engine that enhances care for a patient is a product of training the computer-implemented machine-learning engine. Training of the computer-implemented machine-learning model uses a plurality of training video streams of a corresponding plurality of training patients. Training video data, training audio data, and training semantic text is extracted from a plurality of training video streams of the corresponding plurality of training patients. The extracted training video data are analyzed to identify a first training feature set of video features. The extracted training audio data are analyzed to identify a second training feature set of audio features. The extracted training semantic text data are analyzed to identify a third training feature set of semantic text features. A plurality of known training health outcomes corresponding to each of the plurality of training patients captured in the plurality of training video streams is received in some fashion by the computer-implemented machine-learning engine. General model coefficients of the computer-implemented machine-learning model are determined so as to improve a correlation between a plurality of known training health outcomes and a plurality of training patient health outcomes as determined by the computer-implemented machine-learning model. In some embodiments, model features from the first, second, and third training feature sets are selected as being indicative of the known training healthcare outcomes corresponding to the plurality of training patients captured in the plurality of training video streams. Sometimes some of the first, second, and third training feature sets are selected may not be very indicative of the known healthcare outcomes corresponding to of the plurality of the training patients captured in the plurality of the training video streams. Such not-very-indicative features are sometimes not selected for use in the computer-implemented machine-learning model

While the invention has been described with reference to an example embodiment(s), it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method for enhancing care for a patient, the method comprising:
extracting video data, audio data, and semantic text data from a video stream of the patient with a patient classification;
analyzing the video data to identify a first feature set of video features identified by a computer-implemented machine-learning engine as being indicative of at least one of a set of health outcomes of training patients classified with the patient classification of the patient;
analyzing the audio data to identify a second feature set of audio features identified by the computer-implemented machine-learning engine as being indicative of at least one of the set of health outcomes of the training patients classified with the patient classification of the patient;
analyzing the semantic text data to identify a third feature set of semantic text features identified by the computer-implemented machine-learning engine as being indicative of set of at least one of the set of health outcomes of the training patients classified with the patient classification of the patient;
predicting the predicted health outcome of the patient based on the first, second and/or third features sets, wherein the predicted health outcome of the patient is predicted using a computer-implemented machine-learning model generated by the computer-implemented machine-learning engine;
comparing the predicted health outcome with a set of known training health outcomes of the training patients classified with the patient classification of the patient;
identifying differences between the first, second, and/or third feature sets corresponding to the patient and feature sets of the training patients who have better health outcomes than the predicted health outcome of the patient; and
reporting the differences identified.

2. The method of claim 1, further comprising:
identifying features that are common to the feature sets of the training patients who have better health outcomes than the predicted health outcome of the patient; and
reporting the features identified.

3. The method of claim 1, wherein reporting the differences includes:
automatically reporting the predicted health outcome to a digital medical record associated with the patient.

4. The method of claim 1, wherein reporting the predicted health outcome includes:
automatically reporting the predicted health outcome to a medical doctor who is caring for the patient.

5. The method of claim 1, wherein the computer-implemented machine-learning model has been trained to predict the predicted health outcome, training of the computer-implemented machine-learning model includes:
extracting training video data, training audio data, and training semantic text data from a plurality of training video streams of a corresponding plurality of the training patients;
analyzing the training video data to identify a first training feature set of video features;
analyzing the training audio data to identify a second training feature set of audio features;
analyzing the training semantic text data to identify a third training feature set of semantic text features;
receiving a plurality of known health outcomes corresponding to each of the plurality of training patients captured in the plurality of training video streams; and
determining parameter coefficients of the computer-implemented machine-learning model, such parameter coefficients determined so as to improve a correlation between a plurality of known health outcomes and a plurality of training patient health outcomes as determined by the computer-implemented machine-learning model.

6. The method of claim 5, wherein training of the computer-implemented machine-learning model further includes:
selecting model features from the first, second, and third feature sets, the model features selected as being indicative of known health outcomes corresponding to each of the plurality of training patients captured in the plurality of training video streams.

7. The method of claim 5, wherein the video stream of the patient is added to the plurality of training videos along with a known health outcome of the patient.

8. The method of claim 5, wherein the computer-implemented machine-learning model is a general patient-outcome model, the method further comprises:
identifying a set of outcome-known video portions of the patient, each of the outcome-known video portions capturing features indicative of a known patient outcome;
extracting person-specific video data, person-specific audio data, and person-specific semantic text data from the set of outcome-known video portions of the patient;
analyzing the person-specific video data to identify a first person-specific feature set of video features;
analyzing the person-specific audio data to identify a second person-specific feature set of audio features;
analyzing the person-specific semantic text data to identify a third person-specific feature set of semantic text features; and
determining person-specific parameter coefficients of a person-specific patient-outcome model, such person-specific parameter coefficients determined so as to improve a correlation between the known patient outcomes and patient outcomes as determined by the person-specific patient-behavior model.

9. The method of claim 1, wherein the first feature set includes metrics related to:
a number of times a first video feature occurs;
a frequency of occurrences of the first video feature;
a time period between occurrences of the first video feature; and/or
a time period between occurrences of the first video feature and a second video feature.

10. The method of claim 1, wherein the second feature set includes metrics related to:
a number of times a first audio feature occurs;
a frequency of occurrences of the first audio feature;
a time period between occurrences of the first audio feature; and/or
a time period between occurrences of the first audio feature and a second audio feature.

11. The method of claim 1, wherein the third feature set includes metrics related to:
a number of times a first semantic text feature occurs;
a frequency of occurrences of the first semantic text feature;
a time period between occurrences of the first semantic text feature; and/or
a time period between occurrences of the first semantic text feature and a second semantic text feature.

12. The method of claim 1, further comprising:
generating a fourth feature set that includes feature combinations of at least two of:
a video feature, an audio feature, and a semantic text feature.

13. The method of claim 12, wherein the fourth feature set includes metrics related to:
a number of times feature combination occurs;
a frequency of occurrences of the feature combination;
a time period between occurrences of the feature combination; and/or
a time period between occurrences of a first feature combination and a second feature combination.

14. The method of claim 1, wherein the set of alerting behaviors includes a mental state of the patient, the mental state is based on the first feature set, the second feature set, the third feature set, and a multidimensional mental-state model, wherein:
the multidimensional mental-state model includes a first dimension, a second dimension, and a third dimension;
the first dimension corresponds to a first aspect of mental state;
the second dimension corresponds to a second aspect of mental state; and
the third dimension corresponds to a third aspect of mental state.

15. A system for enhancing care for a patient, the system comprising:
a video camera configured to capture a video stream of a patient;
a processor configured to receive: the video stream of the patient; and
computer readable memory encoded with instructions that, when executed by the processor, cause the system to:
extract video data, audio data, and semantic text data from a video stream of the patient with a patient classification;
analyze the video data to identify a first feature set of video features identified by a computer-implemented machine-learning engine as being indicative of at least one of a set of health outcomes of training patients classified with the patient classification of the patient;
analyze the audio data to identify a second feature set of audio features identified by the computer-implemented machine-learning engine as being indicative of at least one of the set of health outcomes of the training patients classified with the patient classification of the patient;
analyze the semantic text data to identify a third feature set of semantic text features identified by the computer-implemented machine-learning engine as being indicative of set of at least one of the set of health outcomes of the training patients classified with the patient classification of the patient;
predict the predicted health outcome of the patient based on the first, second and/or third features sets, wherein the predicted health outcome of the patient is predicted using a computer-implemented machine-learning model generated by the computer-implemented machine-learning engine;
compare the predicted health outcome with a set of known training health outcomes of the training patients classified with the patient classification of the patient;
identify differences between the first, second, and/or third feature sets corresponding to the patient and feature sets of the training patients who have better health outcomes than the predicted health outcome of the patient; and
report the differences identified.

16. The system of claim 15, further comprising:
identifying features that are common to the feature sets of the training patients who have better health outcomes than the predicted health outcome of the patient; and
reporting the features identified.

17. The system of claim 15, wherein reporting the differences includes:
automatically reporting the predicted health outcome to a digital medical record associated with the patient.

18. The system of claim 15, wherein reporting the predicted health outcome includes:
automatically reporting the predicted health outcome to a medical doctor who is caring for the patient.

19. The system of claim 15, wherein the computer-implemented machine-learning model has been trained to predict the predicted health outcome, training of the computer-implemented machine-learning model includes:
extracting training video data, training audio data, and training semantic text data from a plurality of training video streams of a corresponding plurality of the training patients;
analyzing the training video data to identify a first training feature set of video features;
analyzing the training audio data to identify a second training feature set of audio features;
analyzing the training semantic text data to identify a third training feature set of semantic text features;
receiving a plurality of known health outcomes corresponding to each of the plurality of training patients captured in the plurality of training video streams; and
determining parameter coefficients of the computer-implemented machine-learning model, such parameter coefficients determined so as to improve a correlation between a plurality of known health outcomes and a plurality of training patient health outcomes as determined by the computer-implemented machine-learning model.

20. The system of claim 19, wherein training of the computer-implemented machine-learning model further includes:
selecting model features from the first, second, and third feature sets, the model features selected as being indicative of known health outcomes corresponding to each of the plurality of training patients captured in the plurality of training video streams.
